(19) United States
(12) Patent Application Publication
(10) Pub. No.: US 2022/0023093 A1
(43) Pub. Date: Jan. 27, 2022

(54) DUAL USE INTERORAL PROTECTION MASK

(71) Applicant: Toshio Hosogai, Torrance, CA (US)

(72) Inventor: Toshio Hosogai, Torrance, CA (US)

(21) Appl. No.: 17/362,833

(22) Filed: Jun. 29, 2021

Related U.S. Application Data
(60) Provisional application No. 63/057,250, filed on Jul. 27, 2020.

(51) Int. Cl. A61F 6/04

(52) U.S. Cl. CPC A61F 6/04; A61F 2006/042

(57) ABSTRACT

A protective oral sex face mask is described herein that protects a first wearer and second user during sexual activity. The mask performs as a prophylactic against STDs and other infectious agents. The face mask includes a body portion bounded by a top boundary, a bottom boundary, a first side boundary, and an opposing side boundary. The body portion further includes an interior surface touchable by the first user's tongue, and an exterior surface touchable by a second user. The mask includes a snug tongue pouch maintaining sensory perception of the first wearer while performing oral sex.

### CROSS-REFERENCE TO RELATED APPLICATIONS

This patent application claims priority to U.S. Provisional Application 63/057,250, filed Jul. 27, 2020 the content of which are incorporated herein by reference.

### BACKGROUND OF THE INVENTION

### 1. Field of the Invention

The present invention generally relates to a protective oral face mask to protect the wearer from transmittable diseases, and more particularly, to a protective oral face mask having a pocket indentation in the shape of a human tongue enabling protection for both participants in sexual activity.

### 2. Description of Related Art

Many sexually transmitted diseases (STDs) and viral infections including coronavirus disease (COVID-19) caused by the virus SARS-CoV-2, may be transmitted via oral sex, either by the mouth and tongue or genital engagement. To fight the spread of STDs and viruses, dental dams or other physical barriers are recommended while performing oral sex. However, these conventional barriers may be uncomfortable to wear and hinder the performer's performance. Manufacturers of protective apparatus like these, fail to consider and protect users from kissing, involving lips and tongues of both parties engaged in sexual activity.

Thus, there exists a need for a protective oral sex face mask that is comfortable to wear, pleasing for a wearer to use, and acts as a prophylactic against STDs and other viruses and infectious agents.

### BRIEF SUMMARY OF THE INVENTION

A protective dual use inter-oral protective face mask is provided in one embodiment including a thin and flexible body having a surface area covering at least a first user's mouth. The body may include a top boundary, a bottom boundary, a first side boundary, and an opposing side boundary. In this embodiment, the body portion further includes an interior surface and an exterior surface, the interior surface being touchable by the wearer's tongue and the exterior surface being touchable via a tongue or genital part of a recipient.

In one embodiment an ear loop is provided extending from the first side boundary and the second side boundary. A tongue pouch may be formed in the body enabling the first wearer to insert a tongue wherein material forming the body snugly fits around the tongue of the first user. In this embodiment, the tongue pouch, during use, is enabled to be inserted into the first user's mouth while engaging sexual activity with a second user or extends away from the first user engaging with a second user.

An additional embodiment provides that the body portion is further dimensioned such that the top boundary is wearable beneath the wearer's nose and along the wearer's top lip, where the body portion drapes down across the wearer's lip area, and the bottom boundary runs under the wearer's chin and the body portion, tongue pouch and ear loops are manufactured from an elastic polymeric material. In one embodiment, the elastic polymeric material is latex. Additionally, at least one of a stimulation substance, a lubricant, or a prophylactic substance is administered on the exterior surface of the body portion when in use.

### BRIEF DESCRIPTION OF THE SEVERAL VIEWS OF THE DRAWINGS

FIG. 1 is a front perspective view of a dual-use inter-oral protection mask with a tongue pouch extended outwards.

FIG. 2 is a perspective front view of the mask of FIG. 1 with the tongue pouch inverted from the extended position.

FIG. 3 is an elevated view of a back surface of the mask viewing into the tongue pouch.

FIG. 4 is a view of the mask worn by a user.

### DETAILED DESCRIPTION OF THE INVENTION

The present invention has utility as a protective oral ace mask that is comfortable to wear, pleasing for a wearer to use, and acts as a prophylactic against infectious agents transferred via person-to-person contact. In addition to preventing infectious diseases, it protects non-periodontal people and non-smokers from bad breath caused by periodontal disease patients and smokers.

It should be appreciated that the protective face mask may be used by men and women alike. In preferred embodiments, the face mask is designed for inter-oral kissing, where tongues of parties engaged in kissing may insert tongues into each other's mouth and sexual acts involving a user's tongue, vagina and/or anus.

The face mask is intended for use as a protective medical device to fight the transmission of sexually transmitted disease. Additional features of the mask promote the use of the medical device as further described below. The following description of various embodiments of the invention is not intended to limit the invention to those specific embodiments, but rather to enable any person skilled in the art to make and use this invention through exemplary aspects thereof.

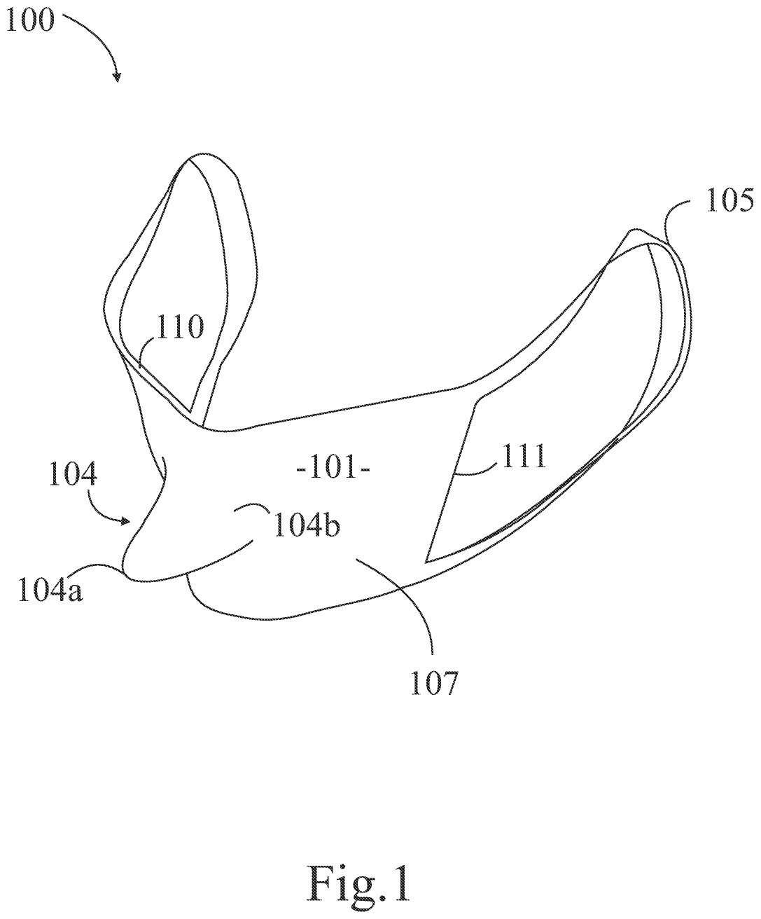

Referring to FIGS. 1 through 4, examples of the dual use inter-oral face mask employing the principles and concepts of the present protective face mask will be described. A preferred embodiment of the present invention includes a dual inter-oral protection mask 100 worn below a wearer's nose and about the wearer's mouth and tongue. In this embodiment a thin, flexible mask body 101, ear loops 105, a chin portion and tongue pouch 104 may be manufactured from polymeric material including very thin latex, nano-cellulose (plant based) material or polyurethane sheets.

An especially novel portion of mask 100 is the formed tongue pouch 104. The human tongue is unique in that it is the only muscle that isn't connected to bone at both ends. It is connected on one end to the hyoid bone, which is also unique as it is the only bone not connected to any other bone in the body. The tongue is very nimble as one may intricately control movement for speech, kissing involving two tongues and other oral sexual activity, including manipulating genitals for sexual stimulation.

The tongue pouch 104 is form fitted for a human tongue which is especially advantageous for specific uses of the mask, as mentioned above. As scan be seen in the figures, tongue pouch 104 includes a diameter at a base, or root 104b where it merges into mask body 101. Tongue pouch 104 also includes a length to accommodate the body of the tongue and an apex 104a extending away from the root 104b. Ear loops 105 are looped around a user's ears to hold the mask in place while in use. Ear loops 105 include a wider portion 105b that engages a user's ears behind the ear to maintain comfort for the user.

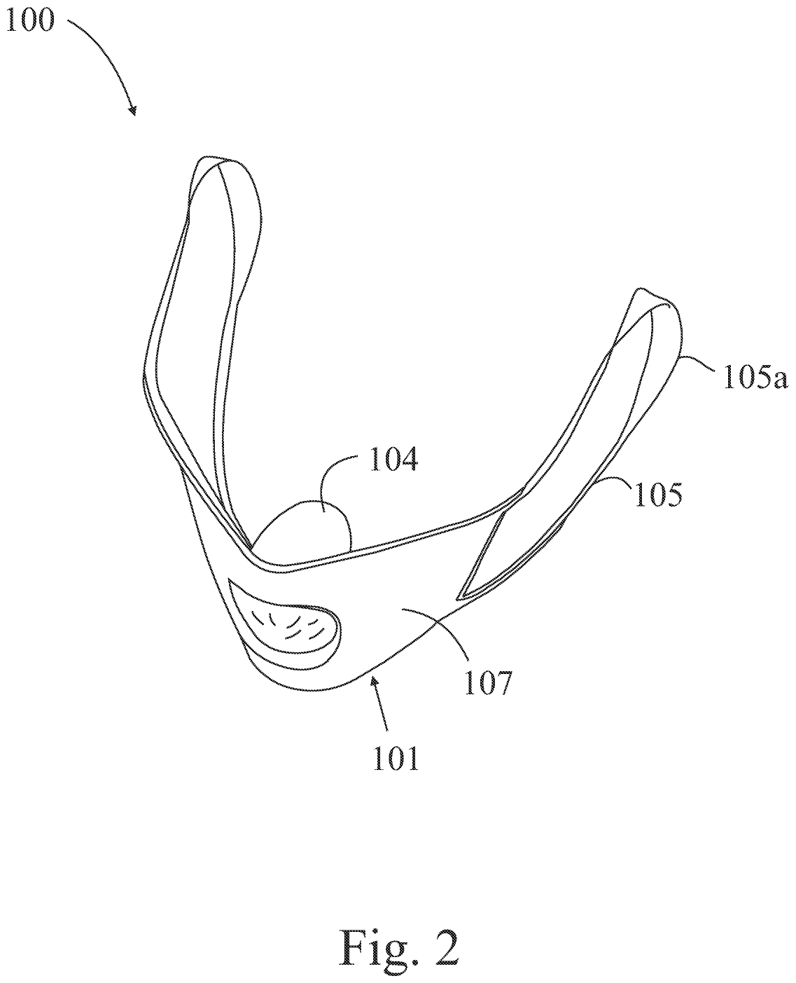

FIG. 2 depicts a mask 100 with tongue pouch 104 inverted. In this view tongue pouch 104 may extend out, away from a face of a user while wearing the mask or invert and be inserted into the user's mouth by a tongue or penis of a second user. In this embodiment an outside surface 107 of mask 100 may directly engage a second user's tongue or penis while being inserted into the user's mouth. The pouch may invert to extend away from the user via the user's tongue in the tongue pouch 104 wherein the pouch 104 is inserted into the second user's mouth or even within a vagina or anus during oral sex.

The inventive pouch 104 of mask 100 is enabled to snuggly fit around the tongue of the first user when extending away from the first user or snuggly fit a tongue of the second users. The material of mask 100 is very thin and flexible enabling almost full sensory perception and movement of the tongues (or penis) of the users during use.

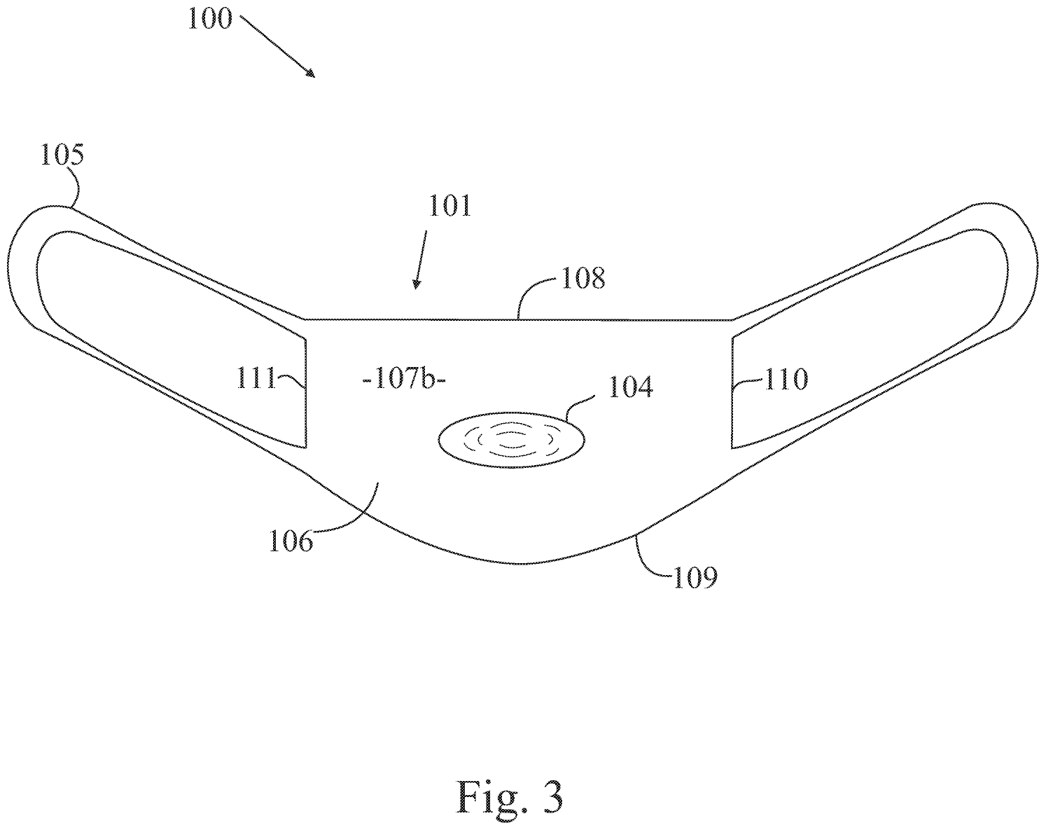

FIG. 3 depicts an interior view of a mask 100 having ear loops 105 to secure the mask 100 to the wearer's face. In one embodiment, the body portion 101 is dimensioned such that the top boundary 108 is wearable beneath the wearer's nose and along the wearer's top lip, and a bottom boundary 109 runs under the wearer's chin as shown in FIG. 3.

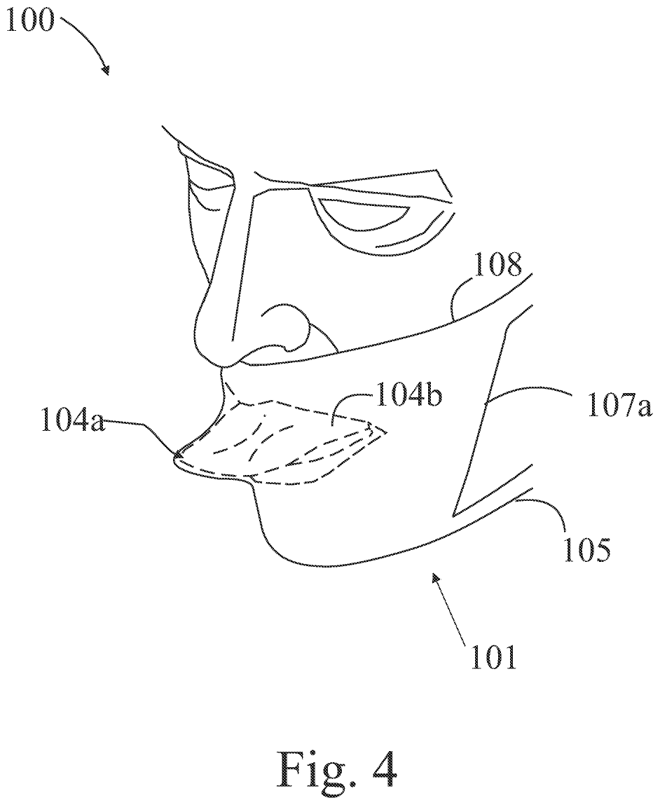

FIG. 4 depicts a user wearing mask 100 with their tongue inserted into tongue pouch 104. As can be clearly seen in FIG. 4, the apex of the tongue fits securely into portion 104a and the root is form fitted at the base 104b. The face mask 100 may be available in different sizes, for example small medium and large, to accommodate differently sized face of the wearer, while in other embodiments the face mask 100 is dimensioned as a one-size-fits-all.

Another embodiment may provide the face mask 100 in two different models, an over-the-nose model with openings at the nose (nostril) area to accommodate breathing and an under the nose model. Nostril openings may be formed with the mask material extending into the nostril or seamed to snugly fit around each nostril in order to provide additional protection. One advantage of mask 100 is all components are integral and manufactured from one contiguous material providing less abrasion and comfort for both users. In one embodiment, the material used to manufacture mask 100 is less than 10 mm, in another it is less than 5 mm, while in a further embodiment it is less than 1 mm, for example as thin as 0.01 mm or less. The thinness may contribute to the wearer's increased sensation enabling the user to move the tongue in more precise ways to stimulate the second user. The face mask 100 may further include a stimulation substance, lubricant, and/or prophylactic substance on the exterior surface 107 of the body portion 101.

In one embodiment a stimulation substance or lubricant may provide sexual stimulation to the recipient or second user during oral sex. A prophylactic substance may further reduce the probably of STD transmission. Examples of stimulation substances include synthetic or natural female/male arousal fluids, gels, or powders. The lubricant may be any safe sexual lubricant known in the art. The prophylactic substance may also include antibiotics or other known infectious agent combatants. The stimulation substance, lubricant, and/or prophylactic substance may be initially covered by a seal that is subsequently removed for use as described above with regard to the flavored substance. Olfactory substances may be added to the mask material, as well, including various flavors and smells including fruit, various flowers, mint, chocolate, vanilla, and strawberry such as ice cream to name a few.

While at least one exemplary embodiment has been presented in the foregoing detail description, it should be appreciated that a vast number of variations exist. It should also be appreciated that the exemplary embodiment or exemplary embodiments are only examples, and are not intended to limit the scope, applicability, or configuration of the described embodiments in any way. It should be understood that various changes may be made in the function and arrangement of elements without departing from the scope as set forth in the appended claims and the legal equivalents thereof.

1. A protective dual use inter-oral protective face mask comprising:
a thin and flexible body including a surface area covering at least a first user's mouth, the body including a top boundary, a bottom boundary, a first side boundary, and an opposing side boundary;
an ear loop extending from the first side boundary and the second side boundary; and
a tongue pouch formed in the body enabling the first wearer to insert a tongue wherein material forming the body snugly fits around the tongue of the first user;
wherein the body supports the tongue pouch and further includes an interior surface and an exterior surface, the interior surface is enabled to be touched by at least the first user's tongue and the exterior surface enabled to be engaged by a second user.

2. The face mask of claim 1, wherein the tongue pouch during use is enabled to be inserted into the first user's mouth while engaging sexual activity with the second user or extends away from the first user.

3. The face mask of claim 1 wherein the body portion is further dimensioned such that the top boundary is wearable beneath the user's nose and along the first user's top lip, where the body portion drapes down across the wearer's lip area, and the bottom boundary runs under the first user's chin.

4. The face mask of claim 1 wherein the body portion, tongue pouch and ear loops are manufactured from an elastic polymeric material.

5. The face mask of claim 3 wherein the elastic polymeric material is latex.

6. The face mask of claim 2 further comprising at least one of a stimulation substance, a lubricant, or a prophylactic substance on the exterior surface of the body and tongue pouch when in use.

7. The face mask of claim 4, wherein the polymeric material has a thickness of between 0.01 mm and 10 mm.
